(19) United States
(12) Patent Application Publication
(10) Pub. No.: US 2026/0115354 A1
(43) Pub. Date: Apr. 30, 2026

(54) HYDROLYZED COLLAGEN COMPOSITIONS FOR CARTILAGE REPAIR AND METHODS OF MAKING AND USING THE SAME

(71) Applicant: Rochal Technologies, LLC, San Antonio, TX (US)

(72) Inventors: Nina Bionda, Ithaca, NY (US); Rebecca E. McMahon, San Antonio, TX (US); Ann Beal Salamone, Gainesville, FL (US); Tiffany F. Sanchez, Helotes, TX (US); Fahimeh Tabatabaei, Boerne, TX (US); Jonathan D. Velasco, Helotes, TX (US); Sergio Medina, San Antonio, TX (US)

(73) Assignee: Rochal Technologies, LLC, San Antonio, TX (US)

(21) Appl. No.: 19/371,938

(22) Filed: Oct. 28, 2025

Related U.S. Application Data
(60) Provisional application No. 63/712,853, filed on Oct. 28, 2024.
(60) Provisional application No. 63/713,305, filed on Oct. 29, 2024.

(51) Int. Cl. A61L 27/24; A61L 15/32; A61L 27/34; A61L 27/52; A61L 27/54

(52) U.S. Cl. CPC A61L 27/24; A61L 15/325; A61L 27/34; A61L 27/52; A61L 27/54; A61L 2430/06

(57) ABSTRACT

Hydrolyzed collagen compositions are described which are used for treatment of impaired cartilage or to prevent damage to healthy cartilage. These compositions function across multiple cartilage components and structures. Oral, topical, or cartilage-site delivery of the compositions to cartilage may be via liquid, gel, powder, or sheet form. Additionally, the hydrolyzed collagen composition may be applied as a coating on a medical device.

### CROSS-REFERENCE TO RELATED APPLICATIONS

This application is related to U.S. Application No. 63/712,853, filed Oct. 28, 2024, and U.S. Application No. 63/713,305, filed Oct. 29, 2024, the entireties of which are incorporated herein by reference.

### FIELD OF INVENTION

This invention relates generally to compositions for treatment of impaired cartilage and for prevention of cartilage damage. In particular, compositions containing hydrolyzed collagen and at least one cartilage support agent are disclosed.

### BACKGROUND

Cartilage is connective tissue that protects joints and bones. It is found in the joints, spine, ribs, neck, bronchial tubes, trachea, ears and nose, for example, of vertebrates. Three types of cartilage exist which vary in function and composition; however, they all are avascular, alymphatic, and aneural. Transport of nutrients and waste is via diffusion to and from adjacent tissues (e.g., bone) and fluids (e.g., synovial fluid). Cartilage is composed of different layers, each serving specific functions including diffusion membranes. Thus, changes in composition related to impaired cartilage impact diffusion and thus, enable certain molecules (e.g., inflammatory proteins, vascular materials) to penetrate through cartilage tissue while detrimentally enabling excessive flow of peptides and proteins out of cartilage.

Damage to cartilage can be painful and even debilitating. A wide range of techniques and compositions have been developed to help maintain and heal cartilage, ameliorate the associated pain, or both.

### SUMMARY OF THE INVENTION

The hydrolyzed collagen compositions described herein provide a combined approach for treating chondrocyte (cartilage), subchondral bone, perichondrium, and matrix stabilization when cartilage is under duress. Such duress can cause intracellular pH imbalances, glucose accumulation, and lipid accumulation, which can lead to excessive reactive ions and collagen/proteoglycan matrix degradation/apoptosis. Metabolic pathway changes (e.g., glycolysis, oxidative phosphorylation, lipid metabolism) from exogenous (trauma) and endogenous (inflammatory disease) events contribute to these factors.

The hydrolyzed collagen compositions described herein include hydrolyzed collagen and at least one cartilage support agent. Each cartilage support agent can independently be selected from a matrix synthesis agent, a pH modulation agent, a glucose modulating agent, and a lipid modulating agent. The cartilage support agents are particularly adapted for cartilage repair and support.

The hydrolyzed collagen composition described herein can be used to reduce or repair cartilage, subchondral bone, and/or perichondrium damage from acute injuries.

The hydrolyzed collagen compositions described herein can be used to reduce or repair damage caused by disease, such as diabetes, arthritis, autoimmune diseases, among others.

The hydrolyzed collagen compositions described herein can be used to prevent cartilage, subchondral bone, and/or perichondrium impairment.

The hydrolyzed collagen compositions described herein can be used to treat cartilage, subchondral bone, and/or perichondrium impacted at surgical incision sites.

The hydrolyzed collagen compositions described herein can be used to treat acute cartilage, subchondral bone, and/or perichondrium impacted wounds.

The hydrolyzed collagen compositions described herein can be used to facilitate tissue healing.

The hydrolyzed collagen compositions described herein can be deposited onto a surface or a device (e.g., an implant) to give enhanced cartilage, subchondral bone, and/or perichondrium repair.

The hydrolyzed collagen compositions described herein can be delivered systemically through oral administration or by injection.

The hydrolyzed collagen compositions described herein may contain proteins and peptides selected from extracellular matrices, glycoproteins, structural proteins, fibrous proteins, enzymes, proteoglycans, natural polypeptides, synthetic polypeptides, globular proteins, membrane proteins, plasma proteins, peptides, oligopeptides, antimicrobial peptides, peptide hormones, chaperones, metalloproteins, hemoproteins, coagulation proteins, immune system proteins, ion channel proteins, cell adhesion proteins, neuropeptides, nucleoproteins, scleroproteins, chromoproteins, conjugated proteins, protein-protein complexes, protein-polysaccharide complexes, protein-lipid complexes, protein-polymer complexes, motor proteins, mucoproteins, phosphoproteins, contractile proteins, transport proteins, signaling proteins, regulatory proteins, growth factors proteins, sensory proteins, defense proteins, storage proteins, receptor proteins, antibodies, recombinant proteins, fibrinogen, fibrin, thrombin, collagen, elastin, albumin, gelatin, keratin, laminin, and combinations thereof.

The hydrolyzed collagen compositions described herein may contain derivatized proteins, which are protein components attached, bound, coordinated, or complexed with another material, such as other proteins, polysaccharides, oligosaccharides, glycosaminoglycans, lipids, phospholipids, liposomes, synthetic polypeptides, DNA, RNA, synthetic polymers, surfactants, metal atoms, nanoparticles, antimicrobial agents, antibiotics, drugs, salts thereof, and the like. In some such embodiments, the protein portion is selected from extracellular matrix and collagen.

The hydrolyzed collagen compositions described herein can include biologically active agents. The biologically active agent can include cells, stem cells, and/or amniotic cells.

In some embodiments, the biologically active agent is tissue or micronized or morselized tissue, such as, but not limited to, spinal cord, bladder, small intestinal submucosa, skin, dermis, epidermis, fat, cartilage, placenta, extracellular matrix, tendon, umbilical cord, cornea, heart, myocardium, liver, pancreas, and muscle.

In some embodiments, the biologically active agent is tissue or micronized or morselized decellularized extracellular matrix derived from cells.

In some embodiments, the biologically active agent is amniotic tissue or morselized amniotic tissue.

In some embodiments, the biological active agent is biologic tissue. In some embodiments, the biologically active agent is minced tissue. In some embodiments, the biologically active agent is micronized tissue. In some embodiments, the biologically active agent is granulated crosslinked bovine tendon collagen and/or glycosaminoglycan.

In some embodiments, the biologically active agent is amniotic fluid. In some embodiments, the biologically active agent is Wharton's jelly.

In some embodiments, the biologically active agent is exosomes. In some embodiments, the biologically active agent is a growth factor.

In some embodiments, the biologically active agent is an analgesic. In some embodiments, the analgesic agent is lidocaine or glucocorticosteroids, such as hydrocortisone.

In some embodiments, the biologically active agent has antimicrobial properties. In some embodiments, the antimicrobial agent is poly(hexamethylene biguanide) and its salts.

In some embodiments, the hydrolyzed collagen compositions can be in the form of powders, tablets, capsules, liquids, gels, creams, lotions, foams, films, sheets, or sprays.

In some embodiments, the hydrolyzed collagen compositions described herein can be coated, injected, sprayed, painted, or implanted in or on tissues, organs, wound void spaces, tissue substitutes, bandages, and medical devices.

In some embodiments, methods of treating cartilage and the associated surroundings, such as bone, meniscus, synovial fluid, tendons, that have been impaired or may become impaired using the hydrolyzed collagen compositions are provided.

In some embodiments, the hydrolyzed collagen composition can be delivered orally, topically, or directly at the site, i.e., cartilage, subchondral bone, and/or perichondrium.

These and other objectives and advantages of the compositions and method described herein, some of which are specifically described and others that are not, will become apparent from the detailed description and claims that follow.

### BRIEF DESCRIPTION OF THE DRAWINGS

FIG. 1 shows a graph of % cell viability determined by Alamar Blue assay of cells treated with hydrolyzed collagen versus a control.

FIG. 2 shows a graph of LDH activity at 2, 5, and 7 days for tissue treated with hydrolyzed collagen versus a control.

FIG. 3 shows a graph of release of cartilage matrix component determined using GAG analysis release at 2, 5, and 7 days for hydrolyzed collagen samples versus control samples.

### DETAILED DESCRIPTION OF THE INVENTION

In this specification and the appended claims that follow, reference will be made to a number of terms that shall be defined to have the following meanings:

It must be noted that, as used in the specification and the appended claims, the singular forms “a”, “an” and “the” include plural references unless the context clearly dictates otherwise. Thus, for example, reference to “a cartilage repair agent” or “cartilage support agent” includes mixtures of two or more such agents, and the like.

As used herein, the term “about” indicates that a value can vary by up to ±5%, ±2%, or ±1%.

As used herein, the term “body” means the physical whole structure of a person or an animal.

As used herein, the phrases “percent solids” and “total solids” have their standard meaning of the quantification of the non-volatile ingredients in a composition based on the solids (e.g., exclusive of the solvent).

As used herein, the phrase “aqueous media” refers to a spectrum of water-based solutions including, but not limited to, homogeneous solutions in water with solubilized components, cell media solutions, buffer solutions, isotonic solutions, salt solutions, emulsified solutions, surfactant solutions, amniotic fluids, Wharton's jelly, serum, hydrophilic polymer solutions, and viscous or gelled homogeneous or emulsified solutions in water.

As used herein, “surfactant” has its standard meaning and includes compounds that lower the surface tension (or interfacial tension) between two liquids or between a liquid and a solid and includes emulsifying agents, emulsifiers, detergents, wetting agents, and surface-active agents.

As used herein, “proteins” is intended to include protein-based macromolecules and includes extracellular matrices, glycoproteins, structural proteins, fibrous proteins, enzymes, proteoglycans, natural polypeptides, synthetic polypeptides, globular proteins, membrane proteins, plasma proteins, antimicrobial proteins, peptide hormones, chaperones, metalloproteins, hemoproteins, coagulation proteins, immune system proteins, ion channel proteins, cell adhesion proteins, neuropeptides, nucleoproteins, scleroproteins, chromoproteins, conjugated proteins, protein-protein complexes, protein-polysaccharide complexes, protein-lipid complexes, protein-enzyme complexes, protein-polymer complexes, motor proteins, mucoproteins, phosphoproteins, contractile proteins, transport proteins, signaling proteins, regulatory proteins, growth factors proteins, sensory proteins, defense proteins, storage proteins, receptor proteins, antibodies, recombinant proteins, fibrinogen, fibrin, thrombin, collagen, elastin, albumin, gelatin, keratin, laminin, and combinations thereof. It should be noted that while collagen is a protein, hydrolyzed collagen is not considered a protein.

As used herein, “derivatized proteins” are protein components attached, bound, coordinated, or complexed with another material, such as other proteins, polysaccharides, oligosaccharides, glycosaminoglycans, lipids, phospholipids, liposomes, synthetic polypeptides, DNA, RNA, synthetic polymers, surfactants, metal atoms, nanoparticles, antimicrobial agents, antibiotics, drugs, salts thereof, and the like.

As used herein, “biologically active agents” has its standard meaning and includes chemical or biological substances or formulations that beneficially affect human or animal health and well-being or is intended for use in the cure, mitigation, treatment, prevention, or diagnosis of infection or disease, or is destructive to or inhibits the growth of microorganisms.

As used herein, “antimicrobial agent” has its standard meaning and include a substance that kills microorganisms or inhibits their growth or replication, while an “anti-infective agent” is defined as a substance that counteracts infection by killing infectious agents, such as microorganisms, or preventing them from spreading. Often, the two terms are used interchangeably.

In one aspect, a hydrolyzed collagen composition including hydrolyzed collagen and at least one cartilage support agent is provided. In some embodiments, the hydrolyzed collagen composition includes hydrolyzed collagen and at least two cartilage support agents. In the hydrolyzed collagen compositions, each cartilage support agent is independently selected from a matrix synthesis agent, a pH modulation agent, a glucose modulation agent, and a lipid modulation agent. The hydrolyzed collagen compositions are specially adapted to help repair and strengthen cartilage and associated tissue (e.g., peri-cartilage).

Cartilage is connective tissue that protects joints and bones. It is found in the joints, spine, ribs, neck, bronchial tubes, trachea, ears and nose of, for example, vertebrates. Three types of cartilage exist which vary in function and composition: hyaline cartilage, fibrocartilage, and elastic cartilage.

Hyaline cartilage, a subset which includes articular cartilage, lines joints and covers bone ends. It has a low coefficient of friction and is generally smooth with some flexibility. Articular cartilage is found at the end of bones that form joints, between ribs and nasal passages, for instance. Of all the cartilage types, articular cartilage has the highest concentration of cells, chondrocytes. The extracellular spaces are composed of collagen II primarily, proteoglycans and glycosaminoglycans. A key function of hyaline cartilage is to resist compressive forces, such as those that occur within knees and hips.

Fibrocartilage is made of thick collagen fibers to form tough tissue that holds muscle and bones together, resists high degrees of tension and compression, and absorbs impact. Fibrocartilage is found in the knee meniscus, spinal discs, and supporting muscles, tendons and ligaments. Fibrocartilage has fewer chondrocytes than other cartilage types; has no chondrocyte pericellular matrix; and is rich in collagen I with notably less proteoglycan than hyaline cartilage.

Elastic cartilage is flexible and allows bending while returning to the original shape once the stress is removed. Elastic cartilage is found in the external part of ears, the Eustachian tubes, the epiglottis, and the larynx. Elastic cartilage contains fewer chondrocytes than hyaline cartilage but more extracellular matrix materials-especially elastin. Proteoglycans and collagen II are key components of its extracellular matrix. Elastic cartilage has a unique outer layer of peri-cartilage containing fibroblast cells on the surface, which transition to chondrocyte cells as the fibroblasts move towards the inner layer of the peri-cartilage.

Hyaline cartilage, which often is impaired due to aging or joint overuse, is of particular concern. The superficial zone of hyaline cartilage is at the synovial fluid interface and serves as a first line of defense for cartilage integrity. Hyaline cartilage is a membrane which blocks large inflammatory proteins from entry while allowing nutrients in and waste to pass out (Adam 2024). Concurrently, it serves as a hard, smooth surface for joint articulation. The chondrocytes are flattened within a laminar matrix of collagen II with pendant lubricin (proteoglycan 4). This construct is similar to a hydrogel as it is hydrated with water. The collagen II is parallel to the cartilage-synovial fluid interface providing a shear resistant sheath.

Beneath this sheath is a middle zone composed of chondrocytes within their lacunae located within a principally non-crystalline matrix of collagen II, hyaluronic acid (glycosaminoglycan) with pendant aggrecan (proteoglycan), other proteoglycans, oligomeric matrix protein (thrombospondin-5), and a small amount of water. This composite serves as a cushioning layer to provide compressive strength as well as a transport membrane and reactive medium for nutrients and waste.

In hyaline cartilage's deep zone, chondrocytes are the most populous and are present with one or two chondrocytes per lacuna. The extracellular matrix is composed of collagen II aligned perpendicularly to the underlying bone and the overlaying middle zone. Proteoglycans and a very small amount of water are present. The lacunae (also known as the pericellular matrix) surround the chondrocytes serving as a nutrient rich environment (including growth factors) while also providing protective compressive strength and serving as a fortress barrier against destructive molecules, such as inflammatory proteins. Experiments have shown that 70 kDa dextran and 500 kDa dextran pass through healthy lacunae at a slower rate than they pass through the middle cartilage zone (Zhang 2015). The lacuna is the first medium for communication between the chondrocytes and the extracellular matrix and plays a key role in cartilage homeostasis. Lacunae are composed of collagen VI as well as collagen II and proteoglycans. Collagen VI is assembled into tetramers intracellularly and transformed within the lacuna into fibrillar aggregates through interaction with a complex of biglycan, decorin, and βig-h3. These collagen VI aggregates are then linked to aggrecan and collagen II forming discreet crystalline domains (aggregates) interconnected with elastic and flexible polymers. This structure provides the pericellular matrix with compressive strength and, hence, a healthy environment for the chondrocytes (Zhang 2015). There are at least 258 genes in the pericellular matrix (lacuna), which up or down regulate with homeostasis intent. Certain basement membrane proteins (perlecan, collagen IV, laminins) are also present closer to the chondrocyte cell rather than distributed throughout the pericellular matrix. While mechanical, chemical and biological interactions discussed here are not exhaustive, they provide context.

Chondrocytes use anaerobic glycolysis to produce the cartilage extracellular matrix and pericellular materials, including glycosaminoglycans, proteoglycans, and collagen. Healthy chondrocytes produce principally collagen II and VI while a signature of unhealthy cartilage is production of collagen I and collagen III, as well as, the transition of chondrocyte to fibroblasts. When glucose and cholesterol metabolisms become dysfunctional, build-up of glucose and lipids occur, and pro-inflammatory and catabolic activities are triggered.

Glucose is the main metabolic fuel for chondrocytes and is transported through cartilage tissue to the chondrocytes via proteins identified as glucose transports (GLUTs). Under stress, chondrocytes signal for more glucose/energy. However, those chondrocytes that cannot adapt to increased glucose levels, may absorb more glucose (Adams 2024) and, as a consequence, osmolality increases, and high levels of reactive ions are generated.

Adult articular cartilage contains lipids (<1%), which are found within the chondrocytes, pericellular matrix and the extracellular matrix. Palmitic, oleic, and linoleic acid distribution profiles remain constant in normal and arthritic cartilage and represent 85% of the total fatty acids in cartilage. However, the amount of total fatty acid increases dramatically with damaged cartilage. More severely impaired cartilage has significantly increased levels of total fatty acid (440%) and essential fatty acids (1,100%). Arachidonic acid accumulation was notable in impaired cartilage constituting 62% of total fatty acids (Lippiello 1991). Reduced levels of high-density lipoproteins (HDL) impair cartilage homeostasis (Adam 2024), possibly due to HDL's function to remove cholesterol, triglycerides, and phospholipid molecules from cells.

With abrupt cartilage damage, such as sports injuries or other traumas, chondrocytes increase metabolism to effect cartilage repair. However, if repair is extensive or the damage is continuous (e.g., osteoarthritis), the chondrocyte hyperactivity becomes destructive resulting in cell apoptosis and matrix degradation.

During early stages of injury, lacunae become larger and more penetrable to increase production and transport of extracellular matrix materials. Perlecan and keratin sulfate are upregulated. Collagen VI is upregulated as well but is aggregated less into crystalline domains and rather found as grouped collagen fibrils indicating dysfunction of the aggregation pathway. This dysfunction creates a stiffer, less flexible matrix which decreases compressive strength and alters communication pathways. Concurrently, chondrocytes increase glycolysis which leads to increasing lactate levels, decreasing ATP generation, and increasing oxidative matrix degradation (Adam 2024).

Lactate accumulation decreases the pH within chondrocytes which under normal homeostasis is 7.5-7.2. This acidic intracellular environment impairs production of extracellular matrix materials (Adam 2024, Browning 2004).

As injury from chronic disease (osteoarthritis) or serious trauma progresses, the pericellular matrix is disrupted and degenerative proteins are released into the inter-territorial matrix (Karim 2018). Pro-inflammatory cytokines, matrix metalloproteases and collagen I are produced with serious cartilage impairment while collagen II and proteoglycan production decline. The effects are increasing inflammatory response, matrix disintegration, and cell apoptosis. With impairment progression, fluid (lipid, water) volume increases, solid volume decreases, and structural composite components (e.g., crystalline domains, elastic and/or flexible networks, communication pathways) disintegrate.

Thus, there is a need for a composition that can counteract these effects of collagen injury, that can also facilitate healing and strengthening of the injured or impaired collagen.

The hydrolyzed collagen compositions described herein include hydrolyzed collagen and at least one cartilage support agent, where each cartilage support agent is independently: a matrix synthesis agent, a pH modulating agent, a glucose modulating agent, or a lipid modulating agent. In some embodiments, hydrolyzed collagen compositions can include hydrolyzed collagen and at least two cartilage support agents. The hydrolyzed collagen composition is used to treat impaired cartilage, subchondral bone, and/or perichondrium and to prevent degradation of cartilage, subchondral bone, and/or perichondrium.

Hydrolyzed collagen is known for its chondroprotective effects and for stimulating chondrocyte production of collagen II for use within the pericellular matrix and the extracellular matrix (Oesser 2003). Hydrolyzed collagen resists proteolytic enzyme and hydrolytic enzyme degradation and readily transports through cartilage tissue to access chondrocytes intracellularly. Hydrolyzed collagen is composed of amino acids, oligomers, and smaller polymers and has a molecular weight range of 50 Da to 12,000 Da. Amino acids typically found in hydrolyzed collagen include, but are not limited to, alanine, arginine, aspartic acid, glutamic acid, glycine, histidine, isoleucine, leucine, lysine, methionine, phenylalanine, proline, hydroxyproline, serine, threonine, tyrosine and valine. Glycine, proline and hydroxyproline are concentration predominant in hydrolyzed collagen. Hydrolyzed collagen is readily water soluble from cold to hot temperatures and does not gel (Bloom strength of zero) except at high % solids (e.g., 60 wt % or higher in aqueous media).

Hydrolyzed collagen may be derived from acid, alkaline, or enzymatic hydrolysis of collagen (molecular weight of about 300 kDa) to produce hydrolyzed collagen (molecular weight of generally less than 12 kDa). The source of collagen can be from human, bovine, porcine, piscine, ovine, or avian sources, but can include other sources and can be a mixture of collagen sources. In some embodiments, the hydrolyzed collagen is present at a concentration ranging from 30 wt % to 99.9 wt %, based on percent solids of the hydrolyzed collagen composition. In some embodiments, the hydrolyzed collagen is present at a concentration ranging from 50 wt % to 99 wt %, or from 60 wt % to 98 wt %.

### Matrix Synthesis Agents

In some embodiments, the hydrolyzed collagen composition includes a matrix synthesis agent.

In some embodiments, the matrix synthesis agent is present at a concentration ranging from 1×10⁻⁹ wt. % to about 50 wt. %, wherein the weight percent is based on total solids. In some embodiments, the hydrolyzed collagen composition comprises at least 0.01 wt. %, at least 0.1 wt. %, or at least 1 wt. % of matrix synthesis agent based on the total solids. In some embodiments, the hydrolyzed collagen composition comprises up to 40 wt. %, up to 30 wt. %, up to 25 wt. %, up to 20 wt. %, or up to 15 wt. % of matrix synthesis agent based on the total solids.

In some embodiments, the matrix synthesis agent includes one or more of the following: amino acid dimers, amino acid trimers, collagen II, IV, or VI, placental tissue, placental fluid, amniotic tissue, amniotic fluid, decorin, cannabidiol, and chlorophyllin.

In some embodiments, amino acid dimers and trimers, which contribute to the synthesis of extracellular matrix collagen, are added to the cartilage-repair hydrolyzed collagen compositions described herein. Pro-Hyp (Pro=L-proline, Hyp=3-hydroxy-L-proline) is an example of an amino acid dimer that increases cellular activity. Gly-Pro-Hyp (Gly=glycine, Pro=L-proline, Hyp=3-hydroxy-L-proline) and other trimers of the structure Gly-X-Y are of interest (Dar 2017, Lee 2021). Gly-Pro-Hyp trimers provide local conformational flexibility, which contributes to maintenance of molecular ordering within collagen fibrils (e.g., collagen II and VI) and, thus, contributes to the structural integrity of cell ligand binding sites (Chow 2018). This function may be a reason for its competency in stimulating chondrocyte production of collagen II and proteoglycans. The addition of up to 10 wt % amino acid trimers to the hydrolyzed collagen composition is preferred. In some embodiments, at least 0.1 wt % amino acid trimers are present in the hydrolyzed collagen composition. Most preferred is the addition of Gly-Pro-Hyp trimer up to 10 wt % based on percent solids of the composition.

In some embodiments, the hydrolyzed collagen composition is blended with collagen. In some embodiments, the hydrolyzed collagen composition comprises at least one of collagen II, collagen IV, and collagen VI. In some embodiments, the hydrolyzed collagen composition comprises each of collagen II, collagen IV, and collagen VI.

Collagen II contains epitopes which are anti-inflammatory and is known to increase proteoglycan content in knee cartilage (Martinez-Puig 2023). Collagen VI and collagen IV, which are present in cartilage compartments, can be supplemented by treatment with these collagens or by treatment with amniotic fluid, placental tissue, umbilical cord tissue, exosomes containing these collagens and combinations thereof. In some embodiments, up to 15 wt % of collagen II, VI, or IV, singly or in combination, based on percent solids, is added to the hydrolyzed collagen composition described herein. In some embodiments, at least 0.1 wt % of collagen II, VI, or IV, singly or in combination, based on percent solids, is added to the hydrolyzed collagen composition described herein.

In some embodiments, the hydrolyzed collagen composition described herein can include placental tissues. Placental tissues are a source of collagen, elastin, fibronectin, and growth factors that can support cartilage repair and regeneration. In particular, amniotic tissue has anti-adhesive and antimicrobial properties, and amniotic tissue has been shown to support tissue repair, reduce inflammation, and minimize scar tissue formation, which are significant benefits in the treatment of impaired cartilage. Thus, the hydrolyzed collagen compositions described herein can include placental tissue in amounts up to 50 wt % based on percent solids. In some embodiments, the hydrolyzed collagen compositions described herein can include placental tissue in amounts of at least 0.1 50 wt % based on percent solids.

In some embodiments, the hydrolyzed collagen composition described herein can include amniotic tissues and fluid. Amniotic tissues and fluid have been described as immune-privileged in that an immune response in the body rarely occurs in response to the introduction of amniotic tissue or fluid. The hydrolyzed collagen compositions described herein can include amniotic tissues and/or amniotic fluid in amounts up to 50 wt % based on percent solids. The hydrolyzed collagen compositions described herein can include amniotic tissues and/or amniotic fluid in amounts of at least 0.1 wt % based on percent solids.

In some embodiments, the hydrolyzed collagen composition described herein can include decorin. Decorin (90-140 kDa), a leucine-rich proteoglycan component of the extracellular matrix, down regulates cancer cell migration and mediates cartilage matrix degradation by increasing the retention of fragmented aggrecan, hence extending mechanical support for the extracellular matrix (Li 2020). In some embodiments, no more than 5% higher than homeostasis levels of decorin are preferred for the cartilage-repair hydrolyzed collagen composition.

Cannabidiol is an anti-inflammatory agent for arthritis. In some embodiments, cannabidiol can be added to the hydrolyzed collagen compositions described herein. Cannabidiol has direct antioxidant properties through reducing pro-oxidant enzyme activity and oxidative modification of lipids, proteins, and DNA. Concurrently, cannabidiol induces transition metal ion chelation, antioxidant enzyme activity, micronutrients to support antioxidant enzymes, and non-enzymatic antioxidants. Cannabidiol's molecular structure has several resonance capabilities as well as multiple sites for amino acid binding. Thus, cannabidiol provides a robust competency to impact multiple biochemical reactions. Up to 5 wt % cannabidiol based on percent solids may be used in the hydrolyzed collagen compositions described herein to mitigate inflammation and, hence, glucose and lipid accumulation in cartilage. In some embodiments, at least 0.1 wt % cannabidiol based on percent solids can be present in the hydrolyzed collagen compositions.

In some embodiments, the hydrolyzed collagen composition described herein can include chlorophyllin. Chlorophyllin, a water-soluble semi-synthetic derivative of chlorophyll, can provide anti-inflammatory properties. In some embodiments, chlorophyllin can be present in an amount up to 5 wt % based on percent solids of the hydrolyzed collagen composition described herein. In some embodiments, chlorophyllin can be present in an amount of at least 0.1 wt % based on percent solids of the hydrolyzed collagen composition described herein.

In some embodiments, the matrix synthesis agent includes one or more of:
0.1 to 10 wt % of amino acid dimers, trimers, or both;
0.1 to 15 wt % of one or more of collagens II, IV, or VI;
0.1 to 50 wt % of placental and/or amniotic tissue and fluid;
1×10⁻⁹ to 0.1 wt-% of decorin;
0.1 to 5 wt % of cannabidiol; or
0.1 to 5 wt % of chlorophyllin, where all percentages are based on total solids.

In some embodiments, the matrix synthesis agent includes one or more of:
0.1 to 10 wt % of Gly-Pro-Hyp;
0.1 to 15 wt % of a combination of collagens II, IV, and VI;
0.1 to 50 wt % of amniotic fluid;
1×10⁻⁹ to 0.1 wt-% of decorin;
0.1 to 5 wt % of cannabidiol; or
0.1 to 5 wt % of chlorophyllin, where all percentages are based on total solids.

In some embodiments, the matrix synthesis agent comprises at least two of the above, or at least three of the above.

### pH Modulating Agents

In some embodiments, the hydrolyzed collagen composition can include a pH modulation agent.

In some embodiments, a pH modulation agent is present at a concentration ranging from about 0.01 wt. % to about 2 wt. %, wherein the weight percent is based on total solids. In some embodiments, a pH modulation agent is present at a concentration of at least 0.05 wt. %, at least 0.1 wt. %, at least 0.15 wt. %, or at least 0.2 wt. %. In some embodiments, a pH modulation agent is present at a concentration of up to up to 1 wt or up to 2 wt %.

In some embodiments, the pH modulation agent comprises at least one of a buffer, copper or a salt thereof, and zinc or a salt thereof.

Intracellularly, chondrocytes rely on Na/H exchange to regulate pH (Browning 2004). Mitochondria produce energy for chondrocytes, which requires an imbalance of protons across the inner mitochondrial membrane to produce energy (ATP). Maintaining an intracellular pH of 7.2-7.5 is vital to mitochondrial energy production, maintenance of reactive ion species, and other functions. During cartilage impairment, this chondrocyte intracellular pH becomes acidic. To mitigate pH drop, buffers can be included as a component of the hydrolyzed collagen compositions described herein. Suitable buffers include phosphate-buffered saline, extracellular saline, Hank's buffered salt solution, Tris-HCl buffer, lactated Ringer's solution, 4-(2-hydroxyethyl) piperazine-1-ethane-sulfonic acid (HEPES), sodium bicarbonate buffers, and mixtures thereof. In some embodiments, one or more buffer dry ingredients can be added to the hydrolyzed collagen composition described herein in an amount ranging from approximately 0.01 to 2.0 wt % by percent solids. In some embodiments, the one or more buffers can be present in an amount ranging from 0.05 to 0.5 wt % based on percent total solids in the composition.

Synovial fluid is also more acidic during impairment, with increased lactic acid playing a role in pH elevation. Zinc and copper and their salts can reduce pH by complexing with lactic acid. Copper, zinc, and their salts are anti-inflammatory and necessary micro-nutrients, but toxic at levels above homeostasis concentration. Zinc also plays a key role in matrix metalloproteinases' function to degrade extracellular matrix components which is important for repair but detrimental with excessive degradation, such as with osteoarthritis and other inflammatory conditions. Therefore, in some embodiments, singly or in combination zinc, copper, and their salts may be incorporated into the hydrolyzed collagen compositions described herein in amounts up to 0.5 wt % based on percent solids of the composition. Suitable copper and zinc salts include those that are water soluble, such as copper and zinc sulfate or chloride, and those salts that are more phospholipid soluble, such as copper and zinc phosphate.

In some embodiments, the hydrolyzed collagen composition includes at least one of the following:
0.1 to 2 wt % of a buffer; or
0.01 to 0.5 wt % of copper or a salt thereof and/or zinc or a salt thereof, where all percentages are based on total solids.

### Glucose Modulating Agents

In some embodiments, the hydrolyzed collagen composition comprises a glucose modulation agent.

In some embodiments, the hydrolyzed collagen composition includes a glucose modulation agent, which is present at a concentration ranging from about 1×10⁻⁹ wt. % to about 30 wt. %, wherein the weight percent is based on total solids. In some embodiments, the glucose modulating agent is present in an amount of at least 0.01 wt. %, at least 0.1 wt. %, or at least 1 wt. %. In some embodiments, the glucose modulating agent is present in an amount of up to 25 wt. %, or up to 20 wt. %, or up to 15 wt. %, or up to 10 wt. %.

In some embodiments, the glucose modulation agent includes at least one of the following: fructose-2,6-bisphosphate, glutamine, glucosamine, and hyaluronic acid.

Phosphofructokinase-2 (PFK-2) produces fructose-2,6-bisphosphate which modulates glucose breakdown through interactions with at least PFK-1. Elevated levels of fructose-2,6-bisphosphate stimulate glucose breakdown by activating PFK-1. Hence, the hydrolyzed collagen composition can include fructose-2,6-bisphosphate in order to reduce glucose levels, which in turn reduces oxidative stress. In some embodiments, levels of fructose-2,6-bisphosphate in the composition are preferred to no more than 5 wt % higher than homeostasis levels.

In some embodiments, the hydrolyzed collagen composition can include glutamine. Glutamine is an L-α-amino acid which has a physiologically neutral pH. It is the most abundant amino acid in the human body and has many functions, one of which is to upregulate heat shock protein 70 (Hsp70), which reduces nitric oxide-induced apoptosis. Glutamine is used by cells as an energy source, in addition to glucose, and glutamine-involved processes are chondro-protective. In some embodiments, glutamine may be present in the hydrolyzed collagen composition described herein at levels of up to 5 weight percent of the percent solids.

In some embodiments, the hydrolyzed collagen composition can include hyaluronic acid. Hyaluronic acid can be used to mitigate osteoarthritis. Hyaluronic acid is a glycosaminoglycan polysaccharide that provides protection against mitochondrial-affected free radical degradation caused by glucose accumulation, among other factors, and prevents apoptosis, and, hence, helps to maintain chondrocyte viability (Grishko 2009).

In some embodiments, the hydrolyzed collagen composition can include glucosamine. Glucosamine can be used to mitigate osteoarthritis due to its function to increase production of hyaluronic acid by synovial cells and chondrocytes. In preferred embodiments, hyaluronic acid or glucosamine may be present in the cartilage-repair hydrolyzed collagen composition at levels of up to 30 wt % based on percent solids.

In some embodiments, the hydrolyzed collagen composition can include one or more of the following:
1×10⁻⁹ to 0.1 wt-% of fructose-2,6-bisphosphate;
0.1 to 5 wt % of glutamine;
1 to 30 wt % of glucosamine; or
1 to 30 wt % of hyaluronic acid, where all percentages are based on total solids.

In some embodiments, the hydrolyzed collagen composition can include fructose-2,6-bisphosphate, hyaluronic acid, or both.

### Lipid Modulating Agents

In some embodiments, the hydrolyzed collagen composition includes a lipid modulation agent.

In some embodiments, the hydrolyzed collagen composition includes a lipid modulation agent at a concentration ranging from about 1×10⁻⁹ wt. % to about 2.5 wt. %, wherein the weight percent is based on total solids. In some embodiments, the hydrolyzed collagen composition includes a lipid modulation agent in an amount of at least 0.0001 wt. %, at least 0.001 wt. %, or at least 0.01 wt. %. In some embodiments, the hydrolyzed collagen composition includes a lipid modulation agent in an amount up to 2 wt. %, up to 1.5 wt. %, up to 1 wt. %, up to 0.1 wt. %, or up to 0.05 wt. %.

In some embodiments, the lipid modulation agent is one or more of the following: high density lipoprotein (HDL), Mito-TEMPO, ω-3 polyunsaturated fatty acids, odd chain saturated fatty acids, and resveratrol and metabolites thereof.

In some embodiments, the hydrolyzed collagen composition includes high density lipoprotein (HDL). HDL carries inflammatory cholesterol, phospholipids, and saturated fatty acids out of chondrocytes and the extracellular matrix, and transports them to the liver for excretion as well as to testes/ovaries for hormone recycling. In some embodiments, incorporating HDL within the hydrolyzed collagen compositions at normal physiological level or slightly higher is preferred.

In some embodiments, the hydrolyzed collagen composition includes Mito TEMPO. Mito TEMPO is a mitochondrial-targeted anti-oxidant that functions by scavenging superoxide and alkyl radicals. Thus, Mito TEMPO can function to reduce oxidative damage within cartilage. Mito TEMPO is a compound composed of piperidine nitroxide and the lipophilic triphenylphosphonium cation (TTP+). Hence, it has lipophilic function for lipid membrane penetration as well as anti-oxidant functionality through the piperidine nitroxide moiety. In some embodiments, Mito TEMPO may be present in the hydrolyzed collagen composition described herein at normal physiological levels or up to 5 wt % higher.

In some embodiments, ω-3 polyunsaturated fatty acids are a component of the hydrolyzed collagen composition described herein. Higher uptake of ω-3 polyunsaturated fatty acid is associated with less cartilage loss which, in part, may be due to stimulation of the production of protectins and resolvins which regulate anti-inflammatory actions (Adam 2024). ω-3 Polyunsaturated fatty acids also mitigate increased fluid uptake within both extracellular matrix and the pericellular matrix and, thus, contribute to reduced hydrostatic pressure on chondrocytes and surrounding tissue. Examples of ω-3 polyunsaturated fatty acids are α-linolenic acid and stearidonic acid as well as eicosapentaenoic acids and docosahexaenoic acids both of which are normally metabolically derived through dietary intake of α-linolenic acid. In some embodiments, the level of ω-3 unsaturated fatty acids is up to 1 wt % based on percent solids of the hydrolyzed collagen composition. In some embodiments, the level of ω-3 unsaturated fatty acids is at least 0.0001 wt % based on percent solids of the hydrolyzed collagen composition.

In some embodiments, the hydrolyzed collagen composition can include one or more odd-chain saturated fatty acids. Addition of odd-chain saturated fatty acids, such as pentadecylic acid and heptadecanoic acid, can decrease progression of osteo arthritis which is postulated to be due to increased fluidity of cell membrane structure (Wu 2017) and, hence, increased cellular signaling and transport of metabolites. Therefore, in some embodiments, odd-chain saturated fatty acids may be present at normal physiological level or no more than 5 wt % higher in the hydrolyzed collagen compositions described herein.

In some embodiments, the hydrolyzed collagen composition can include resveratrol. Resveratrol is a lipid-regulating, anti-inflammatory, and anti-oxidant molecule, which has been found to reduce cholesterol accumulation in cartilage via the SIRT1/FoxO1 pathway (Liang 2023). While Liang's findings used resveratrol with chondrocytes, resveratrol metabolites-resveratrol sulfates and resveratrol glucuronides—are transported to tissues throughout the body. Thus, inclusion of resveratrol can be included in the hydrolyzed collagen compositions described herein. In some embodiments, resveratrol and its sulfate and glucuronide metabolites may be present at levels of up to 1 wt % based on percent solids of the composition. In some embodiments, resveratrol and its sulfate and glucuronide metabolites may be present at levels of at least 0.001 wt % based on percent solids of the composition.

In some embodiments, the hydrolyzed collagen composition includes at least one of the following:
1×10⁻⁹ to 0.1 wt-% of high density lipoprotein (HDL) or Mito TEMPO;
0.0001 to 1 wt % of ω-3 polyunsaturated fatty acids;
1×10⁻⁹ to 1 wt-% of odd chain saturated fatty acids; or
0.001 to 1 wt % of resveratrol and metabolites, where all percentages are based on total solids.

In some embodiments, the hydrolyzed collagen composition comprises a matrix synthesis agent. In some embodiments, the hydrolyzed collagen composition comprises a pH modulating agent. In some embodiments, the hydrolyzed collagen composition comprises a glucose modulating agent. In some embodiments, the hydrolyzed collagen composition comprises a lipid modulating agent.

In some embodiments, the hydrolyzed collagen composition includes 0.0001 to 0.01 wt % of eicosapentaenoic acid.

In some embodiments, the hydrolyzed collagen composition includes HDL and ω-3 polyunsaturated fatty acid.

In some embodiments, the hydrolyzed collagen composition includes pentadecyclic acid, heptadecanoic acid, or both.

In some embodiments, the hydrolyzed collagen composition comprises at least two cartilage support agents. In some embodiments, the hydrolyzed collagen composition comprises at least three cartilage support agents. In some embodiments, the hydrolyzed collagen composition comprises at least four cartilage support agents. In some embodiments, the hydrolyzed collagen composition comprises at least five cartilage support agents. In some embodiments, the hydrolyzed collagen composition comprises at least six cartilage support agents.

In some embodiments, the hydrolyzed collagen composition comprises one cartilage support agent selected from the following: Gly-Pro-Hyp, decorin, cannabidiol, phosphate-buffered saline, fructose-2,6-bisphosphate, HDL, and eicosapentaenoic acid. In some embodiments, the hydrolyzed collagen composition comprises Gly-Pro-Hyp. In some embodiments, the hydrolyzed collagen composition comprises decorin. In some embodiments, the hydrolyzed collagen composition comprises cannabidiol. In some embodiments, the hydrolyzed collagen composition comprises phosphate-buffered saline. In some embodiments, the hydrolyzed collagen composition comprises fructose-2,6-bisphosphate. In some embodiments, the hydrolyzed collagen composition comprises HDL. In some embodiments, the hydrolyzed collagen composition comprises eicosapentaenoic acid.

In some embodiments, the hydrolyzed collagen composition comprises at least two cartilage support agents selected from the following: Gly-Pro-Hyp, decorin, cannabidiol, phosphate-buffered saline, fructose-2,6-bisphosphate, HDL, and eicosapentaenoic acid. In some embodiments, the hydrolyzed collagen composition comprises at least three cartilage support agents selected from the following: Gly-Pro-Hyp, decorin, cannabidiol, phosphate-buffered saline, fructuose-2,6-bisphosphate, HDL, and eicosapentaenoic acid. In some embodiments, the hydrolyzed collagen composition comprises at least four cartilage support agents selected from the following: Gly-Pro-Hyp, decorin, cannabidiol, phosphate-buffered saline, fructuose-2,6-bisphosphate, HDL, and eicosapentaenoic acid. In some embodiments, the hydrolyzed collagen composition comprises at least five cartilage support agents selected from the following: Gly-Pro-Hyp, decorin, cannabidiol, phosphate-buffered saline, fructuose-2,6-bisphosphate, HDL, and eicosapentaenoic acid.

In some embodiments, the hydrolyzed collagen composition includes each of the following: Gly-Pro-Hyp, decorin, cannabidiol, phosphate-buffered saline, fructuose-2,6-bisphosphate, HDL, and eicosapentaenoic acid.

In some embodiments, the hydrolyzed collagen composition includes Gly-Pro-Hyp and eicosapentaenoic acid. In some embodiments, the hydrolyzed collagen composition includes Gly-Pro-Hyp, cannabidiol, and eicosapentaenoic acid.

In some embodiments, the hydrolyzed collagen composition has a form selected from a powder, a liquid, a gel, a paste, a cream, a suspension, an emulsion, a film, a sheet, a foam, a lotion, a spray, an aerosol, a capsule, or a tablet.

In some embodiments, the hydrolyzed collagen composition is a powder which can be preloaded in a syringe, and biologically active agents, such as cells, are drawn into the syringe in order to hydrate the powder before the cell-laden composition is applied (e.g., injected) into or on the body. In other embodiments, dry powder is preloaded in a vial, then the cells in solution are injected through the septum into the vial in order to hydrate the powder, before the cell-laden system is drawn into a syringe and injected into the body. In some embodiments, the hydrated hydrolyzed collagen composition can be applied through a syringe or cannula into or on a body or applied by coating for localized delivery.

In some embodiments, the hydrolyzed collagen composition can include an aqueous solution with one or more of amniotic fluid, morselized amniotic tissue, exosomes, minced tissue, micronized tissue, micronized decellularized tissue, decellularized extracellular matrix derived from cells, granulated cross-linked bovine tendon collagen and glycosaminoglycans, cells and stem cells in cell culture medium, synthetic or naturally derived extracellular matrix components, including collagen, glycosaminoglycans, fibrin, laminin, and fibronectin, hydroxyapatite, honey, polysaccharides, biodegradable polymers, including polyglycolides, polylactides, poly(lactide-co-glycolide), polydioxanone, polycaprolactone, poly(trimethylene carbonate), poly(propylene fumarate), polyurethanes, poly(ester amide)s, poly(ortho ester)s, polyanhydrides, poly(amino acid)s, polyphosphazenes, bacterial polysaccharides and proteins, or combinations thereof, and which can be applied into or on a body.

In some embodiments, the hydrolyzed collagen compositions are hydrated with a fluid. In some embodiments, the mobile phase of the fluid is water, isotonic saline, balanced salt solution, buffer solution, Ringer's solution, cell culture media, stem cell media, serum, plasma, amniotic fluid, Wharton's jelly, nutrient broth, antiseptic solutions, or a combination thereof. In some embodiments, where the fluid is an aqueous media, the aqueous media can have a pH in the range 6.5 to 7.8, most preferably in the range of 7.0-7.4.

In some embodiments, one or more biologically active agents may be incorporated into the hydrolyzed collagen composition to provide a medical benefit to a vertebrate host. Examples of biologically active agents that can be incorporated into the hydrolyzed collagen composition include, but are not limited to, cells, stem cells, amniotic tissue, amniotic cells, exosomes, growth factors, decellularized skin tissue, decellularized extracellular matrix derived from cells, micronized decellularized skin tissue, granulated crosslinked bovine tendon collagen and glycosaminoglycans, antibiotics, antiseptics, anti-infective agents, antimicrobial agents, antibacterial agents, antifungal agents, antiviral agents, antiprotozoal agents, sporicidal agents, antiparasitic agents, peripheral neuropathy agents, neuropathic agents, chemotactic agents, analgesic agents, anti-inflammatory agents, anti-allergic agents, anti-hypertension agents, mitomycin-type antibiotics, polyene antifungal agents, antiperspirant agents, decongestants, anti-kinetosis agents, central nervous system agents, wound healing agents, anti-VEGF agents, anti-tumor agents, escharotic agents, anti-psoriasis agents, anti-diabetic agents, anti-arthritis agents, antipruritic agents, anesthetic agents, anti-malarial agents, anti-arrhythmic agents, anticonvulsants, antiemetic agents, anti-rheumatoid agents, anti-androgenic agents, anthracyclines, anticholinergic agents, anti-aging agents, antihistamines, anti-parasitic agents, hemostatic agents, vasoconstrictors, vasodilators, thrombogenic agents, anticlotting agents, cardiovascular agents, angina agents, sex hormones, growth hormones, isoflavones, integrin binding sequence, biologically active ligands, cell attachment mediators, immunomodulators, tumor necrosis factor alpha, anti-cancer agents, antineoplastic agents, anti-depressant agents, anti-neoplastic agents, narcotic antagonists, anti-hypercholesterolaemia agents, apoptosis-inducing agents, alpha-hydroxyl acids, hormones, tumor-specific antibodies, antisense oligonucleotides, small interfering RNA (siRNA), mRNA, anti-VEGF RNA aptamer, nucleic acids, DNA, DNA fragments, DNA plasmids, transfection agents, vitamins, liposomes, silver nanoparticles, gold nanoparticles, drug-containing nanoparticles, albumin-based nanoparticles, chitosan-containing nanoparticles, polysaccharide-based nanoparticles, dendrimer nanoparticles, phospholipid nanoparticles, iron oxide nanoparticles, bismuth nanoparticles, gadolinium nanoparticles, metallic nanoparticles, ceramic nanoparticles, silica-based nanoparticles, virus-based nanoparticles, virus-like nanoparticles, antibiotic-containing nanoparticles, nitric oxide-containing nanoparticles, nanoshells, nanorods, polymeric micelles, silver salts, zinc salts, quantum dots nanoparticles, polymer-based microparticles, polymer-based microspheres, drug-containing microparticles, drug-containing microspheres, antibiotic-containing microparticles, antibiotic-containing microspheres, antimicrobial microparticles, antimicrobial microspheres, salicylic acid, benzoyl peroxide, 5-fluorouracil, nitroglycerin, clonidine, estradiol, testosterone, fentanyl, diclofenac, buprenorphine, bupivacaine, ketoprofen, opioids, cannabinoids, enzymes, enzyme inhibitors, oligopeptides, cyclopeptides, polypeptides, proteins, derivatized proteins, protease inhibitors, cytokines, chondroitin sulfate, dermatan sulfate, parasympatholytic agents, chelating agents, lipids, glycolipids, glycoproteins, endocrine hormones, growth hormones, growth factors, differentiation factors, heat shock proteins, immunological response modifiers, saccharides, polysaccharides, insulin and insulin derivatives, steroids, corticosteroids, and non-steroidal anti-inflammatory drugs or similar materials, in either their salt form or their neutral form, either being inherently hydrophilic or encapsulated within a hydrophilic microparticle or nanoparticle. Such biologically active agents could be in either of the (R)-, (R, S)-, or(S)-configuration, or a combination thereof.

In some embodiments the hydrolyzed collagen composition may include chondrocytes, chondrons, type A synovial tissue cells, type B synovial tissue cells, phagocytes, or combinations thereof.

In some embodiments the hydrolyzed collagen composition may include adult stem cells, embryonic stem cells, amniotic stem cells, induced pluripotent stem cells, fetal stem cells, tissue stem cells, adipose-derived stem cells, bone marrow stem cells, mesenchymal stem cells, perinatal stem cells, satellite cells, side population cells, multipotent stem cells, totipotent stem cells, unipotent stem cells, or combinations thereof.

In some embodiments, the hydrolyzed collagen composition includes at least one of the following: cells, stem cells, amniotic tissue, amniotic cells, exosomes, growth factors, micronized decellularized skin tissue, decellularized extracellular matrix derived from cells, granulated collagen, gelatin, or glycosaminoglycans. In some embodiments, the cells are animal cells. In some embodiments, the cells are mammalian cells. In some embodiments, the cells are non-mammalian cells.

In some embodiments, the hydrolyzed collagen compositions described herein may be used to deliver a therapeutically effective amount of between 10,000 cells to about 1 billion cells or more.

In some embodiments, the hydrolyzed collagen composition includes at least one additional ingredient selected from glycolipids, glycoproteins, immunological response modifiers, saccharides, steroids, and polysaccharides.

In some embodiments, the hydrolyzed collagen composition can include at least one anti-infective agent. In some embodiments, the hydrolyzed collagen compound includes at least one anti-infective agent including, but are not limited to, biguanides, such as poly(hexamethylene biguanide) (PHMB) and its salts, chlorhexidine and its salts, such as chlorhexidine digluconate, and alexidine and its salts, such as alexidine dihydrochloride, benzalkonium chloride, benzethonium chloride, cetyltrimethylammonium bromide, and capryl glycol. In some embodiments, the hydrolyzed collagen composition includes at least one anti-infective agent including PHMB, capryl glycol, or both.

In some embodiments, an anti-infective biological agent is added to the hydrolyzed collagen composition. The anti-infective can include, but are not limited to, antibiotics, antiseptics, anti-infective agents, antimicrobial agents, antibacterial agents, antifungal agents, antiviral agents, antiprotozoal agents, sporicidal agents, and antiparasitic agents. In some embodiments, the biological agent is biodegradable, non-cytotoxic to human and animal cells, or both biodegradable and non-cytotoxic.

Additional examples of anti-infective agents that can be included in the hydrolyzed collagen compositions include, but are not limited to, biguanides, such as poly(hexamethylene biguanide) (PHMB) and its salts, a low molecular weight synthetic cationic biguanide polymer, chlorhexidine and its salts, such as chlorhexidine digluconate, and alexidine and its salts, such as alexidine dihydrochloride, where the latter two are bis(biguanides), benzalkonium chloride, benzethonium chloride, cetyltrimethylammonium bromide, capryl glycol, iodine, povidone-iodine, starch-iodine, neomycin sulfate, polymyxin B, bacitracin, tetracyclines, clindamycin, nitrofurazone, mafenide acetate, silver nanoparticles, silver sulfadiazine, silver nitrate, terbinafine hydrochloride, miconazole nitrate, ketoconazole, clotrimazole, itraconazole, metronidazole, antimicrobial peptides, polyquaternium-1, polyquaternium-6, polyquaternium-10, salts thereof, and combinations thereof.

As used herein, “antimicrobial agent” has its standard meaning and includes a substance that kills microorganisms or inhibits their growth or replication, while an “anti-infective agent” is defined as a substance that counteracts infection by killing infectious agents, such as microorganisms, or preventing them from spreading. Often, the two terms are used interchangeably.

In some embodiments, an antimicrobial agent is added to the hydrolyzed collagen composition to hinder development and proliferation of microorganisms. In some embodiments, the addition of an antimicrobial agent helps reduce or eliminate microbial colonies and biofilm formation. Because of the possibly of infection in the area of application, the hydrolyzed collagen composition can include a biological agent in an amount sufficient to hinder or eradicate microorganisms. In some embodiments, antimicrobial agents can be added to the cartilage-repair hydrolyzed collagen compositions in concentrations from 0.001 wt to 2.0 wt % based on solids content of the composition.

In some embodiments, an analgesic biological agent is added to the hydrolyzed collagen composition which includes, but is not limited to, lidocaine, glucocorticosteroids, non-steroidal anti-inflammatory drugs (NSAIDs), Cox-2 inhibitors, opioids, acetaminophen, and combinations thereof.

In some embodiments, the cartilage-repair hydrolyzed collagen compositions can have an osmolality of 10-900 mOsm/kg. In some embodiments where the composition is an aqueous-based solution, gel, paste, emulsion, or foam, a water-soluble polymer can be added to increase solution viscosity and to prolong residence time on the applied surface into or on a body. In some embodiments, useful water-soluble polymers include, but are not limited to, poly(ethylene glycol), poly(ethylene oxide), poly(vinyl alcohol) and copolymers, poly(N-vinylpyrrolidone) and copolymers, methylcellulose, hydroxyethylcellulose, hydroxypropylcellulose, hydroxypropylmethylcellulose, carboxymethylcellulose, guar gum, hydroxyethylguar, hydroxypropylguar, gelatin, albumin, hydroxypropylmethylguar, carboxymethylguar, carboxymethylchitosan, locust bean gum, carrageenan, xanthan gum, gellan gum, pullulan, alginate, chondroitin sulfate, dextran, dextran sulfate, Aloe vera gel, scleroglucan, schizophyllan, gum arabic, tamarind gum, poly(methyl vinyl ether), ethylene oxide-propylene oxide-ethylene oxide block copolymers, hyaluronan, chondroitin sulfate, keratan sulfate, dermatan sulfate, heparan sulfate, dextran, carbomer and its salts, poly(acrylic acid) and its salts, poly(methacrylic acid) and its salts, poly(ethylene-co-acrylic acid), poly(vinyl methyl ether), poly(vinylphosphoric acid) salts, poly(vinylsulfonic acid) salts, sodium poly(2-acrylamido-2-methylpropanesulfonate), polyacrylamide(s), poly(N,N-dimethylacrylamide), poly(N-vinylacetamide), poly(N-vinylformamide), poly(2-hydroxyethyl methacrylate), poly(glyceryl methacrylate), poly(2-ethyl-2-oxazoline), poly(N-isopropylacrylamide) and poly(N-vinylcaprolactam), the latter two hydrated below their Lower Critical Solution Temperatures, polyquaternium-1, polyquaternium-6, polyquaternium-10, ionene polymers, cationic guar, pyridinium polymers, imidazolium polymers, diallyldimethylammonium polymers, poly(L-lysine), acryloyl-, methacryloyl-, and styryl-trimethylammonium polymers, acrylamido- and methacrylamido-trimethylammonium polymers, antimicrobial peptides, and the like, and derivatives and combinations thereof.

In some embodiments, the preparation of the hydrolyzed collagen composition can be in the form of viscous solutions, gels, creams, pastes, emulsions, balms, and sprays, can be facilitated by the inclusion of water-soluble polymer viscosity builders in amounts ranging from about 0.01 to about 50.0 wt %, from 0.1 to 45% wt, from 0.5 to 25 wt %, or from 1.0 to 10.0 wt %.

In some embodiments, the hydrolyzed collagen composition can also include wetting agents, gelling agents or emulsifiers. Other excipients that can be added to the hydrolyzed collagen compositions include various surfactants, silicones, polyether copolymers, vegetable and plant fats and oils, hydrophilic and hydrophobic alcohols, vitamins, monoglycerides, laurate esters, myristate esters, palmitate esters, and stearate esters.

In some embodiments, one or more observational or detectable agents may be incorporated into the hydrolyzed collagen composition to provide enhanced visualization or facilitate proper placement. The agents may comprise, in other embodiments, dyes, fluorescent substances, ultraviolet absorbers, radioactive substances, pigments, or any combinations thereof.

In some embodiments, the hydrolyzed collagen composition can be delivered in different forms. Examples of these forms include, but are not limited to, powder, sheet, film, liquid, solutions, gel, suspension, paste, cream, lotion, emulsion, spray, foam, aerosols, capsules, tablets and combinations thereof, and the like. The compositions may be imbibed by biodegradable matrices, or by swabs, sponges, foams, and dressing materials. The composition of this invention may be coated on medical devices, such as joint replacement parts and medical hardware (e.g., screws and plates).

In some embodiments, the cartilage-repair hydrolyzed collagen composition can be delivered via oral, topical, cartilage-site, subchondral bone site, or perichondrium site methods.

### Methods of Treating Tissue.

In another aspect, a method of treating tissue comprising contacting tissue with a hydrolyzed collagen composition described herein.

In some embodiments, the tissue is cartilage, subchondral bone, or perichondrium having a disease or disorder.

In some embodiments, the tissue is cartilage, subchondral bone, or perichondrium and is treated to prevent irritation, damage, disease, or disorder.

In some embodiments, the composition is applied topically, via injection, or via ingestion.

In some embodiments, the injection is an intra-articular, intra-subchondral bone, intra-perichondrium, or intra-cartilage injection, and the tissue comprises cartilage.

As used herein, “topically” has its standard meaning (e.g., directly onto tissue) and includes applying onto a surface of cartilage (e.g., during a surgical intervention).

In some embodiments, the injection can be intradermal, subcutaneous, oral, intramuscular, submucosal, intranasal, vaginal, buccal, intrathecal, epidural, intraparenchymal, ocular, subretinal, dental, intra-tumoral, intracardiac, intra-articular, intravenous, intracavernous, intraosseous, intraperitoneal, intra-abdominal, intra-fascial, intra-organ, and intravitreal.]

### EXPERIMENTAL

The following materials and abbreviations are used in the experimental section.

| Tradename | Chemical | Manufacturer | Lot |
|---|---|---|---|
| Cartilage | lyophilized bovine | Articular Engineering | B-1271 |
| | cartilage powder | | |
| Peptiplus XB | bovine hydrolyzed | Gelita | 8623297 |
| | collagen | | |
| Porcine HC | porcine hydrolyzed | Rochal Technologies | 70-45 |
| | collagen, pH 7 | | |
| UC-II | chicken collagen type II | NOW | 3413533 |
| | and seaweed | | |
| Hemp Oil | cannabidiol | GORNVB | 8162025 |
| Chlorophyllin | 33% chlorophyllin with | Life Extension | L01571E |
| | microcrystalline cellulose, | | |
| | silica, vegetable stearate | | |
| Glutamine | 90% glutamine with | VYKEE Nutrition | 0194-01-004 |
| | magnesium stearate | | |
| Chitosan | chitosan | Aldrich | MKBK4182V |
| Hyaluronic Acid | hyaluronic acid | Sunlab | 14419 |
| Resveratrol | 96% resveratrol with rice | aSquared Nutrition | 200325 |
| | flour | | |
| DPBS | Dulbecco's phosphate | VWR | 22K2956965 |
| | buffered saline | | |
| Grassfed Beef | trachea and scapula | Primal | 120425042027 |
| Bioactive | collagen with bioactive | | |
| Collagen = GBBC | peptides, chondroitin | | |
| | sulfates | | |
| Omega 3 Fish Oil | eicosapentaenoic acid, | Nature's Bounty | 5210UM0730 |
| | docosahexaenoic acid, | | |
| | other fatty acids | | |

### Example 1: Hydrolyzed Collagen with Cartilage Support Agents

The compositions of Table 1, below, were formulated by mixing hydrolyzed collagen (Peptiplus XB or porcine HC) with cartilage support agents and other additives. When mixed, most of the compositions were slight yellow powders. Chlorophyllin with hydrolyzed collagen was a dark gray powder. Peptiplus XB (bovine hydrolyzed collagen) with hemp oil (cannabidiol) created an immediate gel. Each of these compositions could be applied to and spread across cartilage, impaired cartilage, subchondral bone, and perichondrium.

TABLE 1
Hydrolyzed collagen compositions

| | | | | | | | wt % | | |
|---|---|---|---|---|---|---|---|---|---|
| | Hydrolyzed | Amount | Cartilage | Amount | wt % | wt % | Other | Other | |
| Sample | Collagen | (g) | Support Agents | (g) | HC | Agent | Additives | Additives | Observations |
| 1 | Peptiplus XB | 6.506 | Water | 3.501 | 100% | N/A | N/A | N/A | Slight yellow uniform powder |
| 2 | Peptiplus XB | 9.004 | UC-II | | 90% | 5% | N/A | N/A | Slight yellow uniform powder |
| | | | GBBC | | | 5% | | | |
| 3 | Peptiplus XB | 9.504 | Hemp Oil | 0.505 | 95% | 5% | N/A | N/A | Slight yellow uniform |
| | | | | | | | | | powder, slight clumping |
| 4 | Peptiplus XB | 8.312 | Chlorphyllin | 0.556 | 83% | 6% | 11% | Microcrystalline | Dark gray uniform powder |
| | | | | | | | | Cellulose | |
| | | | | | | | | Silica | |
| | | | | | | | | Vegetable Stearate | |
| 5 | Peptiplus XB | 9.443 | Glutamine | 0.504 | 94% | 5% | 1% | Magnesium Stearate | Slight yellow uniform powder |
| 6 | Peptiplus XB | 9.905 | Hyaluronic Acid | 0.103 | 99% | 1% | N/A | N/A | Slight yellow uniform powder |
| 7 | Peptiplus XB | 9.891 | Resveratrol | 0.106 | 99% | 1% | 0.04% | Rice Flour | Slight yellow uniform powder |
| 8 | Peptiplus XB | 6.502 | Hemp Oil | 0.502 | 65% | 5% | N/A | N/A | Immediate gel with |
| | | | DPBS | 3.000 | | 30% | | | components |
| 9 | Peptiplus XB | 9.505 | Omega 3 fish oil | 0.401 | 96% | 4% | N/A | N/A | Slight yellow uniform powder |
| 10 | Porcine HC | 4.758 | Hemp Oil | 0.256 | 95% | 5% | N/A | N/A | Slight yellow uniform |
| | | | | | | | | | powder, slight clumping |
| 11 | Peptiplus XB | 5.077 | Hemp Oil | 5.004 | 50% | 50% | N/A | N/A | Immediate gel with |
| | | | | | | | | | components |
| 12 | Peptiplus XB | 1.609 | UC-II | 0.051 | 80% | 2.5% | 15% | Chitosan | Slight yellow uniform powder |
| | | | GBBC | 0.051 | | 2.5% | | | |
| 13 | Peptiplus XB | 1.642 | Hyaluronic Acid | 0.010 | 82% | 0.5% | N/A | N/A | Slight yellow uniform |
| | | | Hemp Oil | 0.050 | | 2.5% | | | powder, slight clumping |
| | | | DPBS | 0.301 | | 15% | | | |
| 14 | Peptiplus XB | 2.353 | Resveratrol | 0.011 | 78% | 0.3% | 0.01% | Rice Flour | Slight yellow uniform |
| | | | Hemp Oil | 0.050 | | 1.7% | 10% | Chitosan | powder, slight clumping |
| | | | DPBS | 0.301 | | 10% | | | |
| 15 | Peptiplus XB | 2.639 | Hyaluronic Acid | 0.0104 | 88% | 0.34% | 0.01% | Rice Flour | Slight yellow uniform |
| | | | Resveratrol | 0.0106 | | 0.35% | | | powder, slight clumping |
| | | | Hemp Oil | 0.0512 | | 1.7% | | | |
| | | | DPBS | 0.3004 | | 9.98% | | | |

### Example 2: Gels of Hydrolyzed Collagen with Cartilage Support Agents

In the compositions of Table 2, below, water was added to each of the above samples to create gels that were 65 wt % solids. Osmolality was determined using water dilution of the gels to 10 wt % solids. Three osmolality readings were conducted with the average and standard deviation recorded. pH was measured on the 65 wt % solids gels. The gels had a pH range of 6.45 to 7.34. The osmolality had a small range of from 73 to 106. Samples 8Gel and 13Gel containing hydrolyzed collagen and hemp oil formed gels that did not flow at room temperature. Each of these compositions could be applied to and spread across cartilage, impaired cartilage, subchondral bone, and perichondium.

TABLE 2
Hydrolyzed collagen gel compositions

| Sample | pH (65% | Osmolality | Standard | Gel Observations |
|---|---|---|---|---|
| 1Gel | 6.45 | 81.67 | 0.58 | Clear yellow gel, slowly flowable at RT |
| 2Gel | 6.85 | 97.00 | 2.00 | Opaque yellow gel w/ undissolved solids, slowly flowable at RT |
| 3Gel | 6.46 | 76.33 | 0.58 | Slightly opaque yellow gel, slowly flowable at RT |
| 4Gel | 6.86 | 89.33 | 1.53 | Very dark green gel, almost black, w/ particulates, slowly flowable at RT |
| 5Gel | 6.49 | 98.67 | 0.58 | Slightly opaque yellow gel w/ undissolved solids, slowly flowable at RT |
| 6Gel | 6.51 | 94 | 1 | Clear yellow gel, very sticky and stringy, not flowable at RT |
| 7Gel | 6.50 | 80 | 1.73 | Clear golden brown gel, slowly flowable at RT, insoluble particulates. |
| 8Gel | 6.55 | 84 | 1 | Very slightly opaque yellow gel, not flowable at RT |
| 9Gel | 6.48 | 77.00 | 2 | Opaque yellow gel, slowly flowable at RT |
| 10Gel | 7.34 | 94.00 | 1 | Opaque goldish yellow gel, not flowable at RT |
| 11Gel | 6.59 | 61.67 | 6.35 | Swollen HC particles, visible phase separation, yellow color, flowable at RT |
| 12Gel | 7.30 | 73.00 | 2 | Opaque goldish yellow gel with undissolved particulates, not flowable at RT |
| 13Gel | 6.63 | 105.67 | 3.06 | Very slightly opaque yellow gel, not flowable at RT |
| 14Gel | 7.10 | 90.33 | 1.15 | Opaque golden brown gel, slowly flowable at RT |
| 15Gel | 6.39 | 83.70 | 1.2 | Opaque goldish yellow, slowly flowable, very tacky, some particul ates |

### Example 3: Compositions as Coatings on Implantable Materials

Table 3, below, lists samples where the gels of Example 2 were lightly coated on stainless steel and polypropylene coupons as well as a glass surface. As the coated gels dried, they all became tacky with apparent stringiness.

TABLE 3
Gel coatings on stainless steel, polypropylene and glass surfaces

| Sample | Observations |
|---|---|
| 1Gel | Uniform coat, doesn't flow off, slightly tacky |
| 2Gel | Insoluble particulates, doesn't flow off, slightly tacky |
| 3Gel | Uniform coat, doesn't flow off, slightlytacky |
| 4Gel | Some visible particulates, doesn't flow off, tacky |
| 5Gel | Insoluble particulates, doesn't flow off, slightly tacky |
| 6Gel | Uniform coat, doesn't flow off, very sticky and stringy |
| 7Gel | Insoluble particulates, doesn't flow off, slightly tacky |
| 8Gel | Uniform coat, doesn't flow off, slightly tacky |
| 9Gel | Uniform coat, doesn't flow off, slightly tacky |
| 10Gel | NOT TESTED |
| 11Gel | Phase separated mixture, flows off surfaces |
| 12Gel | Some visible particulates, doesn't flow off, slight tack, |
| | no apparent phase separation |
| 13Gel | Uniform coat, doesn't flow off, very tacky |
| 14Gel | Some visible particulates, doesn't flow off, tacky, |
| | no apparent phase separation |
| 15Gel | Mostly uniform coating, some particulates, doesn't flow off, |
| | no apparent phase separation |

### Example 4: Hydrolyzed Collagen Applied to Collagen Sheet Dressing

Samples 3 and 6 from Table 1 and 3Gel and 6Gel from Table 2 were applied to Covalon ColActive Plus which is a collagen matrix dressing in sheet form. The dressing was cut to 1″×1″ size. The cut dressing was coated with 0.1 gram of each powder sample, 3 and 6. The powder was easily spread across the dressing surface but did not adhere to the dressing surface. Additional collagen sheet dressings were coated with 0.5 grams of each gel sample, 3Gel and 6Gel. The gels easily spread across the collagen sheet surface, were adherent to it, and provided adhesion of the cellulose sheet to stainless steel. Absorption of the gels into the cellulose sheets increased the sheet flexibility.

### Example 5: Hydrolyzed Collagen Composition with Cartilage

Hydrolyzed collagen (Peptiplus XB) was mixed with cannabidiol (hemp oil) and bovine cartilage powder at a weight ratio of 90:5:5. The bovine cartilage powder was prepared by lyophilization and milling of cartilage. The composition was a slightly yellow powder with some clumping. When water was added to this powder to create a gel (65 wt % solids), the pH was a biocompatible 6.55 and a biocompatible osmolality of 723±9.5 mOsm/kg for the gel (65 wt % solids). The gel containing cartilage powder was opaque yellow, slightly flowable at room temperature, slightly stringy, and slightly tacky. Thus, this composition could be applied to and spread across cartilage, impaired cartilage, subchondral bone, and perichondrium.

### Example 6: Ex Vivo Bovine Cartilage Model-Effects of Hydrolyzed Collagen on the Cell Viability and Tissue Matrix

To evaluate the effects of hydrolyzed collagen on viable bovine cartilage explants, three readouts were performed: cell viability (AlamarBlue), cytotoxicity (lactate dehydrogenase [LDH] assay, Roche lot No. 11644793001), and extracellular matrix integrity (glycosaminoglycan [GAG] content; DMMB assay Blyscan Sulfated Glycosaminoglycan Assay Kit #B1000).

Viable bovine cartilage explants (purchased from Articular Engineering) were placed into a 96 well-plate. Explants were equilibrated in chondrocyte growth medium with 10% fetal bovine serum (FBS) overnight at 37° C. under 5% CO₂. The medium was then removed and 200 μL of hydrolyzed collagen (30 mg/mL in medium) was added to the tissue samples. Control samples were treated with 200 μL of phosphate buffered saline (PBS). Tissue was incubated at 37° C. under 5% CO₂ for the duration of the experiment and media refreshed every 2-3 days. On days 2, 5, and 7, media was collected for LDH and GAG analysis. Cell viability was evaluated using Alamar Blue assay. The data was normalized to total tissue weight or baseline signal where applicable.

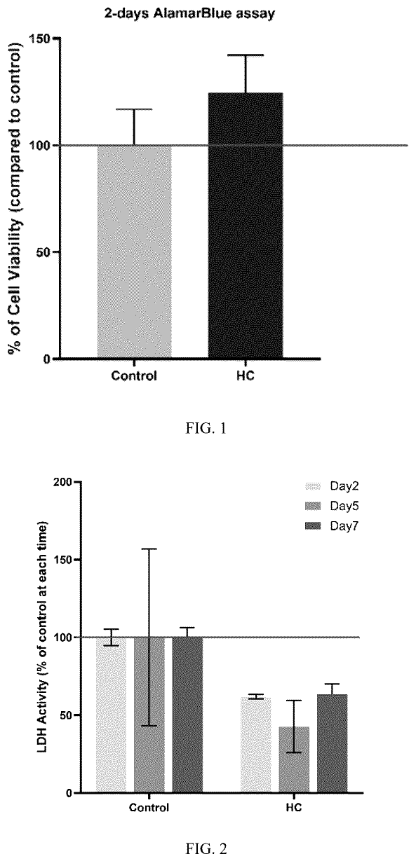

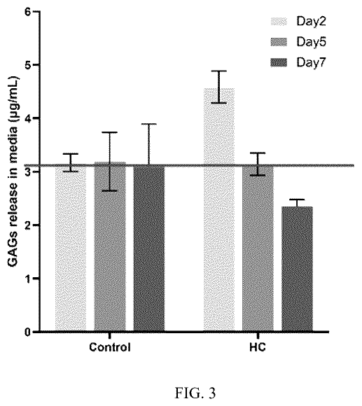

As shown in FIG. 1, cell viability, as determined on day 2 by Alamar Blue assay, showed approximately 25% higher values for hydrolyzed collagen-treated samples compared to untreated control. As shown in FIG. 2, the LDH activity, indicative of cell damage/lysis, was lower for the tissue treated with hydrolyzed collagen for all data points evaluated. As shown in FIG. 3, GAG analysis showed a marked decrease in the release of cartilage matrix components over time, even though at day 2 the GAG release was approximately 45% higher for the hydrolyzed collagen-treated samples than for the control samples. This data demonstrates that hydrolyzed collagen has a positive effect on cartilage tissue and improved cell and tissue matrix over untreated controls.

While the above specification contains many specifics, these should not be construed as limitations on the scope of the invention, but rather as examples of preferred embodiments thereof. Many other variations are possible. Accordingly, the scope of the invention should be determined not by the embodiments illustrated, but by the appended claims and their legal equivalents.

What is claimed is:

1. A composition comprising hydrolyzed collagen and at least one cartilage support agent, wherein each cartilage support agent is independently selected from a matrix synthesis agent, a pH modulation agent, a glucose modulation agent, or a lipid modulation agent.

2. The composition of claim 1, wherein the hydrolyzed collagen is present in an amount ranging from 30 wt % to 99.9 wt % based on the total solids, wherein the weight percent is based on total solids.

3. The composition of claim 1, wherein at least two cartilage support agents are present, and each cartilage support agent is independently selected from a matrix synthesis agent, a pH modulation agent, a glucose modulation agent, or a lipid modulation agent.

4. The composition of claim 1, wherein a matrix synthesis agent is present.

5. The composition of claim 4, wherein the matrix synthesis agent is present at a concentration ranging from 1×10⁻⁹ wt. % to about 50 wt. %, wherein the weight percent is based on total solids.

6. The composition of claim 4, wherein the matrix synthesis agent is selected from the group consisting of amino acid dimers, amino acid trimers, collagen II, IV, or VI, placental tissue, placental fluid, amniotic tissue, amniotic fluid, decorin, cannabidiol, and chlorophyllin.

7. The composition of claim 1, wherein a pH modulation agent is present.

8. The composition of claim 7, wherein a pH modulation agent is present at a concentration ranging from about 0.01 wt. % to about 2 wt. %, wherein the weight percent is based on total solids.

9. The composition of claim 7, wherein the pH modulation agent is selected from the group consisting of a buffer, copper or a salt thereof, and zinc or a salt thereof.

10. The composition of claim 1, wherein a glucose modulation agent is present.

11. The composition of claim 10, wherein a glucose modulation agent is present at a concentration ranging from about 1×10⁻⁹ wt. % to about 30 wt. %, wherein the weight percent is based on total solids.

12. The composition of claim 10, wherein the glucose modulation agent is selected from the group consisting of fructose-2,6-bisphosphate, glutamine, glucosamine, and hyaluronic acid.

13. The composition of claim 1, wherein a lipid modulation agent is present.

14. The composition of claim 13, wherein a lipid modulation agent is present at a concentration ranging from about 1×10⁻⁹ wt. % to about 2.5 wt. %, wherein the weight percent is based on total solids.

15. The composition of claim 13, wherein the lipid modulation agent is selected from the group consisting of high density lipoprotein (HDL), Mito-TEMPO, ω-3 polyunsaturated fatty acids, odd chain saturated fatty acids, and resveratrol and metabolites thereof.

16. The composition of claim 1, wherein the at least one cartilage support agents comprises at least one of the following: Gly-Pro-Hyp, decorin, cannabidiol, phosphate-buffered saline, fructuose-2,6-bisphosphate, HDL, and eicosapentaenoic acid.

17. The composition of claim 1, further comprising a biologically active agent selected from cells, stem cells, amniotic tissue, amniotic cells, exosomes, growth factors, decellularized skin tissue, decellularized extracellular matrix derived from cells, micronized decellularized skin tissue, granulated crosslinked bovine tendon collagen and glycosaminoglycans, antibiotics, antiseptics, anti-infective agents, antimicrobial agents, antibacterial agents, antifungal agents, antiviral agents, antiprotozoal agents, sporicidal agents, antiparasitic agents, peripheral neuropathy agents, neuropathic agents, chemotactic agents, analgesic agents, anti-inflammatory agents, anti-allergic agents, anti-hypertension agents, mitomycin-type antibiotics, polyene antifungal agents, antiperspirant agents, decongestants, anti-kinetosis agents, central nervous system agents, wound healing agents, anti-VEGF agents, anti-tumor agents, escharotic agents, anti-psoriasis agents, anti-diabetic agents, anti-arthritis agents, anti-itching agents, antipruritic agents, anesthetic agents, anti-malarial agents, dermatological agents, anti-arrhythmic agents, anti-convulsants, antiemetic agents, anti-rheumatoid agents, anti-androgenic agents, anthracyclines, anti-smoking agents, anti-acne agents, anticholinergic agents, anti-aging agents, antihistamines, anti-parasitic agents, hemostatic agents, vasoconstrictors, vasodilators, thrombogenic agents, anti-clotting agents, cardiovascular agents, angina agents, erectile dysfunction agents, sex hormones, growth hormones, isoflavones, integrin binding sequences, biologically active ligands, cell attachment mediators, immunomodulators, tumor necrosis factor alpha, anti-cancer agents, anti-depressant agents, antitussive agents, anti-neoplastic agents, narcotic antagonists, anti-hypercholesterolemia agents, apoptosis-inducing agents, birth control agents, emollients, alpha-hydroxyl acids, manuka honey, topical retinoids, hormones, tumor-specific antibodies, antisense oligonucleotides, small interfering RNA (siRNA), mRNA, anti-VEGF RNA aptamer, nucleic acids, DNA, DNA fragments, DNA plasmids, Si-RNA, transfection agents, vitamins, essential oils, liposomes, exosomes, silver nanoparticles, gold nanoparticles, drug-containing nanoparticles, albumin-based nanoparticles, chitosan-containing nanoparticles, polysaccharide-based nanoparticles, dendrimer nanoparticles, phospholipid nanoparticles, iron oxide nanoparticles, bismuth nanoparticles, gadolinium nanoparticles, metallic nanoparticles, ceramic nanoparticles, silica-based nanoparticles, virus-based nanoparticles, virus-like nanoparticles, nitric oxide-containing nanoparticles, nanoshells, nanorods, polymeric micelles, quantum dots nanoparticles, polymer-based microparticles, polymer-based microspheres, drug-containing microparticles, drug-containing microspheres, salicylic acid, benzoyl peroxide, 5-tluorouracil, nicotinic acid, nitroglycerin, clonidine, estradiol, testosterone, nicotine, motion sickness agents, scopolamine, fentanyl, diclofenac, buprenorphine, bupivacaine, ketoprofen, opioids, cannabinoids, enzymes, enzyme inhibitors, proteins, prodrugs, protease inhibitors, hyaluronic acid, chondroitin sulfate, dermatan sulfate, para-sympatholytic agents, hair growth agents, lipids, glycolipids, glycoproteins, endocrine hormones, growth hormones, growth factors, differentiation factors, heat shock proteins, immunological response modifiers, saccharides, polysaccharides, insulin and insulin derivatives, steroids, corticosteroids, and non-steroidal anti-inflammatory drugs or similar materials, in either their salt form or their neutral form, either being inherently hydrophilic or encapsulated within a hydrophilic microparticle or nanoparticle.

18. The composition of claim 1, further comprising at least one of cells, stem cells, amniotic tissue, amniotic cells, exosomes, growth factors, micronized decellularized skin tissue, decellularized extracellular matrix derived from cells, granulated collagen, gelatin, or glycosaminoglycans.

19. The composition of claim 1, further comprising at least one additional ingredient selected from glycolipids, glycoproteins, immunological response modifiers, saccharides, steroids, polysaccharides, or an anti-infective agent.

20. The composition of claim 1, wherein the composition has a form selected from a powder, a liquid, a gel, a paste, a cream, a suspension, an emulsion, a film, a sheet, a foam, a lotion, a spray, an aerosol, a capsule, or a tablet.

21. The composition of claim 1, wherein the composition comprises a fluid selected from the group consisting of water, isotonic saline, balanced salt solution, buffer solution, Ringer's solution, cell culture media, stem cell media, serum, plasma, amniotic fluid, Wharton's jelly, nutrient broth, antiseptic solutions, and combinations thereof.

22. A method of treating tissue comprising contacting the tissue with a composition of claim 1.

23. The method of claim 22, wherein the tissue is cartilage, subchondral bone, or perichondrium.

24. The method of claim 22, wherein the composition is applied topically, via injection, or via ingestion.
